(19) United States
(12) Patent Application Publication
(10) Pub. No.: US 2015/0141332 A1
(43) Pub. Date: May 21, 2015

(54) METHODS FOR DIAGNOSING OSTEOARTHRITIS

(71) Applicant: Biomet Biologics, LLC, Warsaw, IN (US)

(72) Inventor: Krista Toler, Pierceton, IN (US)

(73) Assignee: Biomet Biologics, LLC, Warsaw, IN (US)

(21) Appl. No.: 14/405,726

(22) PCT Filed: Jun. 4, 2013
(86) PCT No.: PCT/US2013/044075

§ 371 Date: Dec. 4, 2014

Related U.S. Application Data
(60) Provisional application No. 61/655,322, filed on Jun. 4, 2012.

(51) Int. Cl. G01N 33/68

(52) U.S. Cl. CPC G01N 33/6863; G01N 2800/105; G01N 2333/49; G01N 2333/485; G01N 2800/52
USPC 514/8.2; 514/8.6; 435/7.92; 514/9.6; 514/7.6; 435/7.94; 506/9

(57) ABSTRACT

Methods for managing osteoarthritis, in a human or other mammalian subject, comprising the measurement of certain cytokines and growth factors in a tissue sample of a subject, including one or more of platelet-derived growth factor AB (PDGF-AB), platelet-derived growth factor BB (PDGF-BB), and epidermal growth factor (EGF). Tissue samples may be whole blood, blood fractions, urine, saliva, and synovial fluid. Methods include diagnosing osteoarthritis, and methods for assessing the severity of osteoarthritis, such as in subject that have been diagnosed with osteoarthritis using radiographic or other methods. Methods may also include comparison of measured cytokine levels to a reference level. Methods of managing the clinical progression of osteoarthritis include initiating a clinical action based on the difference between the measured cytokine level and a reference level.

### BACKGROUND

Osteoarthritis is a potentially debilitating disease that is primarily associated with joint pain. It may also be characterized by joint tenderness, swelling stiffness, muscle weakness, and loss of flexibility. Unlike rheumatoid arthritis, osteoarthritis is not a systemic disease; rather, symptoms of osteoarthritis are usually localized to the affected joint. Severe cartilage loss in the joint can result in misshaping of the joint, which can cause joint instability.

There are currently no common treatment options exist that act to retard or prevent its progression. Although it has been studied to a great extent, there is still no clear answer as to what initiates osteoarthritis, and factors such as aging, joint overuse, obesity, and heredity can all play a role in the onset of the disease. Furthermore, it has not been determined if osteoarthritis is a single disease or a combination of several diseases that have closely related pathways.

Osteoarthritis is currently diagnosed by x-radiographic methods, such as by observation of joint space narrowing. Unfortunately, this diagnosis does not happen until after the patient has already started to lose cartilage content, resulting in the joint space narrowing. A number of inflammatory cytokines such as interleukin-1β (IL-1β) and tumor necrosis factor-α (TNF-α) are known to play a pivotal role in the development of osteoarthritis. However, there are countless numbers of growth factors in human and animal blood. The concentrations of those growth factors can fluctuate depending on a number of factors. Thus, it is extraordinarily difficult to use the measurement of any of these factors as the basis for diagnosing the presence of osteoarthritis in any given clinical subject.

### SUMMARY

The present technology provides methods for managing osteoarthritis, in a human or other mammalian subject. The methods comprise the measurement of certain cytokines and growth factors in a tissue sample of a subject, including one or more of platelet-derived growth factor AB (PDGF-AB), platelet-derived growth factor BB (PDGF-BB), and epidermal growth factor (EGF). Tissue samples may be selected from the group consisting of whole blood, blood fractions, urine, saliva, synovial fluid, and combinations thereof. Such methods include those for diagnosing osteoarthritis, and methods for assessing the severity of osteoarthritis, such as in subject that have been diagnosed with osteoarthritis using radiographic or other methods.

In some embodiments, methods further comprise comparing the concentration of cytokine to a cytokine reference level. The cytokine reference level may be a mean or median value of tissue sample concentrations of the cytokine in a population of the mammalian subjects, such as a population of subjects not having osteoarthritis. The reference level may be the concentration of the cytokine in tissue sample obtained from the subject at a time prior to the time of the measuring.

In some embodiments, methods comprise
(a) measuring the level of a cytokine in a first sample of blood obtained from the subject;
(b) measuring the level of the cytokine in a second sample of blood obtained from the subject; and
(c) comparing the level of cytokine in the first sample with the level of cytokine in the second sample to determine a cytokine level change; wherein
(d) the cytokine is selected from the group consisting of platelet-derived growth factor AB (PDGF-AB), platelet-derived growth factor BB (PDGF-BB), epidermal growth factor (EGF), and combinations thereof.
   Such methods may further comprise:
(i) conducting a first radiographic, or other non-PDGF- or EGF-based diagnostic, assessment of a joint of the subject at the time of measuring the level of the cytokine in the first sample;
(ii) conducting a second radiographic, or other non-PDGF- or EGF-based diagnostic, assessment of the joint of the subject at the time of measuring the level of the cytokine in the second sample;
(iii) comparing the first assessment to the second assessment to determine a radiographic change; and
(iv) correlating the radiographic change to the cytokine level change.
   The present technology also provides a method of managing the clinical progression of osteoarthritis in a mammalian subject, comprising:
(a) measuring the concentration, in the blood of the subject, of a cytokine selected from the group consisting of platelet-derived growth factor AB (PDGF-AB), platelet-derived growth factor BB (PDGF-BB), epidermal growth factor (EGF), and combinations thereof;
(b) comparing the concentration of the cytokine to a cytokine reference level to determine a cytokine level variance; and
(c) initiating a clinical action based on the cytokine level variance.
   The clinical action may be selected from the group of initiating administration of a treatment regimen, modifying the timing of a treatment regimen, modifying the level of a treatment regimen, terminating a treatment regimen, and combinations thereof. The treatment regimen may comprise administration of an anti-inflammatory composition comprising interleukin-1 receptor antagonist (IL-1ra) and soluble tumor necrosis factor receptor I.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

### Methods of Assessment

The present technology provides methods for assessing osteoarthritis in a mammalian subject. Such methods include methods of diagnosing osteoarthritis, assessing the susceptibility of developing osteoarthritis, and monitoring the progression of osteoarthritis, in human and other mammalian subjects. Other mammalian subjects include companion, working, or sports animals, such as dogs, cats and horses.

Traditional radiographic indicators of osteoarthritis are insensitive and may reveal the presence of the osteoarthritis only after significant progression of the disease. Diagnosing early stages of osteoarthritis using such methods of the art is problematic due to difficulty in defining and observes radiographic changes in subjects. The methods of the present technology allow for diagnosing osteoarthritis at developmental and advanced stages. The concentration of platelet-derived growth factor AB (PDGF-AB), in particular, correlates with osteoarthritis indicators in osteoarthritis patients, including joint space narrowing, presence of osteophytes, subchondral sclerosis and subchondral cysts. The following table shows the exemplary average PDGF-AB, PDGF-BB and EGF concentrations in subjects presenting with none, one, two or three of the osteoarthritis indicators:

| | Average | Average | Average |
|---|---|---|---|
| # of | PDGF-AB | PDGF-BB | EGF |
| Osteoarthritis | Concentration | Concentration | Concentration |
| Indicators | (ng/ml) | (ng/ml) | (pg/ml) |
| 0 | 22.5 | 6.7 | 452 |
| 1 | 18.7 | 6.0 | 450 |
| 2 | 15.7 | 4.8 | 350 |
| 3 | 14.5 | 4.9 | 306 |

By monitoring changes in basal levels of various cytokines and growth factors in blood or other tissue samples, early detection of osteoarthritis can be determined. If performed on a subject early enough, osteoarthritis can be detected before it is radiographically evident. Therefore, prevention or slowing of its progression can be accomplished. Early detection of osteoarthritis allows for medical intervention before the disease state is significant.

In various embodiments, the method may be repeated over a course of time, i.e. weeks, months or years, to establish baseline levels of cytokines and growth factors. Changes in these levels indicate the onset of osteoarthritis. Such methods are of particular benefit to subjects at risk of developing osteoarthritis, such as subjects having inherent environmental risks of developing osteoarthritis. Examples of such risks include obesity, genetic predisposition, and previous joint trauma.

Although all patients that have pain associated with osteoarthritis exhibit radiographic evidence of the disease, not all patients that exhibit radiological evidence of the disease have pain. Advantageously, the methods described herein provide a diagnostic tool that provides separate indicators for osteoarthritis risk and severity from pain or radiographic evidence. Therefore, the diagnostic methods allow for a personalized clinical action or treatment. For example, the disease progression can be tracked, a determination can be made as to whether a clinical action is working, and adjustments to the clinical action can be made accordingly. For severe stages of osteoarthritis, aggressive therapies, or multiple therapies can be administered to the patient, as further described below. Less aggressive therapies can be administered to patients at a less severe stage.

Methods for assessing osteoarthritis in a mammalian subject comprise measuring, in a tissue sample of the subject, the concentration of a cytokine selected from the group consisting of platelet-derived growth factor AB (PDGF-AB), platelet-derived growth factor BB (PDGF-BB), epidermal growth factor (EGF), and combinations thereof. It is understood that the measurement may then be used in the assessment of osteoarthritis, and management of the disease, in any manner consistent with good clinical practice by a physician or other health care provider. In some embodiments, a method for assessing osteoarthritis in a mammalian subject comprises measuring a tissue level of a cytokine and comparing the level to a cytokine reference level, wherein a measured level deviating from the reference level indicates the presence of osteoarthritis, susceptibility to arthritis, or progression of arthritis in the subject.

Examples of tissues that can be used for measuring the tissue level of a cytokine include blood, urine, saliva, and synovial fluid. The term “blood” is understood to include whole blood and fractions of whole blood, such as serum, plasma, platelet-rich plasma, and platelet-poor plasma.

In various embodiments, the cytokine reference level is correlated with an indicator of osteoarthritis using second diagnostic (i.e., a diagnostic not employing measurement of PDGF-AB, PDGF-BB and EGF). Such a diagnostic may include a x-ray radiographic indicator, such as joint space narrowing, the presence of osteophytes, subchondral sclerosis, subchondral cysts, or combinations thereof. The cytokine reference level may act as a threshold level which is indicative of further steps in the diagnosis or treatment of the subject. Such steps may include performing additional diagnostics, further monitoring of the subject (such as repeated testing using methods of this technology or other methods over a period of time), or initiating a treatment regimen or other clinical action as discussed below.

In some embodiments, the cytokine reference level reflects the tissue concentrations of the cytokine in a population of the mammalian subjects or in the subject undergoing the measurement. For example, the cytokine reference level may be the mean value, or median value, of the cytokine in the tissue sample (e.g., blood levels) in a population of mammalian subjects not having osteoarthritis (i.e., in subjects of the same species as the subject of the method). For example, a PDGF-AB reference level can be a whole blood concentration of from about 6 ng/ml to about 17 ng/ml, a PDGF-BB reference level can be a whole blood concentration of fro about 2 ng/ml to about 7 ng/ml, and an EGF reference level can be a whole blood concentration of from about 110 pg/ml to about 420 pg/ml. It is understood that such reference levels are subject to statistical variance, and that the threshold value for further steps in diagnosis or treatment may reflect a statistical standard deviation from the reference level. For example, the threshold value may be equal to the mean concentration of the cytokine in a population of subjects measured in a clinical study, plus a concentration equal to the twice the standard deviation of the measurements in the study. In some method, further steps for diagnosis or treatment may be performed if two or more of PDGF-AB, PDGF-BB and EGF are less than threshold values, such as the values set forth above.

In some embodiments, the cytokine reference level is the concentration of the cytokine in tissue obtained from the subject at a time prior to the time of the measuring. Thus, the method may comprise
(a) measuring the level of a cytokine in a first sample of blood obtained from the subject;
(b) measuring the level of the cytokine in a second sample of blood obtained from the subject; and
(c) comparing the level of cytokine in the first sample with the level of cytokine in the second sample to determine a cytokine level change; wherein
(d) the cytokine is selected from the group consisting of platelet-derived growth factor AB (PDGF-AB), platelet-derived growth factor BB (PDGF-BB), epidermal growth factor (EGF), and combinations thereof.

The time prior to the time of measuring (i.e., the time between the measuring in a first sample and the time of measuring in the second sample) can be from days to weeks to months to years. For example, the time prior may be about 1 week, about 2 weeks, about 3 weeks, about 4 weeks, about 6 weeks, about 2 months, about 3 months, 4 months, about 6 months, about 9 months, about 1 year, about 1.5 years, about 2 years, or about 5 years. In various embodiments, the time prior to the time of measuring is at least four weeks, at last six months, or at least about one year. By using a cytokine reference level specific to the subject, small changes in the subject's cytokine profile can be established. These small changes can be used to diagnose an onset of osteoarthritis or monitor the progression of osteoarthritis. When changes indicate the onset of osteoarthritis, the subject can be treated to prevent or slow progression of the disease.

In some methods, further steps for diagnosis or treatment may be performed if PDGF-AB is less than a threshold value, such as a value of from about 2 to about 32 ng/ml; from about from about 5 to about 30 ng/ml, from about 6 to about 17 ng/ml, from about 8 to about 23 ng/ml, or from about 16 to about 26 ng/ml. For example, the threshold PDGF-AB value may be about 1.9, 2, 4.9, 5, 6, 8, 8.2, 13, 13.8, 14, 14.2, 14.5, 14.7, 15, 15.7, 16, 16.6, 16.7, 17, 18.7, 19, 20, 22.5, 23, 23.4, 23.8, 24, 25.6, 26, 31, 31.1, 31.7, or 32 ng/ml. In some methods, further steps for diagnosis or treatment may be performed if PDGF-BB is less than a threshold value, such as a value of about 1 to about 16 ng/ml; about 2 to about 11 ng/ml, about 2 to about 10 ng/ml, or about 2 to about 7 ng/ml. For example, the threshold PDGF-BB value may be about 1, 1.6, 1.9, 2, 3.7, 4, 4.8, 4.9, 5, 5.6, 6, 6.4, 6.6, 6.7, 6.8, 7, or 9, 9.1, 8.6, 9, 10, 11, 11.1, 16, or 16.1 ng/ml. In some methods, further steps for diagnosis or treatment may be performed if EGF is less than a threshold value, such as a value of about 87 to about 1300 pg/ml; about 130 to about 780 pg/ml, about 175 to about 770 pg/ml, about 185 to about 700 pg/ml, or from about 110 to about 420 pg/ml. For example, the threshold EGF value may be about 87, 90, 110, 112, 130, 175, 180, 190, 300, 306, 310, 350, 400, 415, 418, 420, 425, 430, 450, 452, 470, 476, 480, 565, 570, 600, 730, 820, 824, or 830 pg/ml.

Although it may be desired for a subject to establish a cytokine reference level prior to the onset of osteoarthritis, the cytokine reference level can be established in a subject that has symptoms of osteoarthritis, which allows for monitoring progression of the disease. Alternatively, the cytokine reference level can be established prior to the onset of symptoms, and the measuring can be performed when symptoms arise. Non-limiting examples of symptoms of osteoarthritis include joint pain, joint stiffness, joint injury, radiographic anomaly, and combinations thereof. By monitoring changes in cytokine levels relative to the cytokine reference level, a clinical action of the subject can be tailored to the indicated stage of osteoarthritis.

The cytokine reference level can be established before or after the subject is diagnosed with osteoarthritis. For example, when a subject's cytokines are measured after a diagnosis of osteoarthritis, progression of the disease can be monitored. In various embodiments, the severity of osteoarthritis in the subject can be determined, and a severity index can be established. Therefore, the current method can comprise comparing the concentration of a cytokine in the tissue of the subject to a cytokine reference level, and determining a severity index value based on the result of the comparison. In some embodiments, the method further comprises conducting radiographic assessments of a joint of the subject at the time of establishing the cytokine reference level, and/or at the time of measuring the concentration of the cytokine in the tissue. Comparing the radiographic assessments can be used to determine a radiographic change, which can be correlated to the cytokine level change. These changes can influence the clinical action to be performed on the subject.

### Methods of Measurement

Measuring a tissue level of a cytokine may be performed using any method suitable for measuring the levels of cytokines in blood or other tissue samples. Such methods include bioassays and immunoassays known in the art. Measurement may be performed by obtaining a blood or other tissue sample in a clinical setting (e.g., in a physician's office or hospital room) and testing in a laboratory. In some embodiments, the testing may be performed at the point-of-care, using a suitable diagnostic test device. Testing may be performed by the subject of the present methods, or by a physician, nurse, or other health care provider.

In various embodiments, measurement comprises use of an enzyme-linked immunosorbent assay (ELISA). ELISA assays are typically performed in a multi-well assay plate. Various embodiments are modeled from a sandwich ELISA in which multiple small strips can be used in place of an assay plate. A strip would comprise an end composed of a material that is transparent to visible light, such as polystyrene or methacrylate. The end would comprise a small well, a plurality of wells, or an immobilization section on which antibodies can be bound. The small well(s) or immobilization section can be coated with antibodies specific for a cytokine. A user could dip the end of the strip into a blood or plasma sample that contains the cytokine of interest. Strips could also be dipped into solutions of various standard concentrations of the cytokine. After incubating, the strips can be washed. The ends of the strips could then be dipped into a solution comprising a second antibody specific for the cytokine that is conjugated to an enzyme, such as horseradish peroxidase (HRP). Unbound antibody-HRP conjugates are then washed out of the well(s) or immobilization section. The end of the strips are then dipped into a substrate solution comprising tetramethylbenzidine (TMP), water, and H₂O₂. The strips can then be inserted into a reader that geometrically accepts the strips so an absorbance reading can be made at the well(s) or immobilization section at a wavelength of 450 nm.

Another embodiment is an ELISA assay performed on a V-chip as described by Song, et al., Nat Commun. 2012; 3:1283. Doi: 10.1038/ncomms2292. V-chips are microfluidic chips comprising a plurality of wells and channels, and two plates. The V-chips have a top end and a bottom end. When one plate is moved relative to the second plate, flow channels are formed. Therefore, a variety of components can be preloaded into a V-chip via flow lanes before a reaction occurs. A top flow lane is preloaded with ink or dye. The flow lane second from the top is precoated with anti-cytokine antibodies and then loaded with a plasma sample containing the analyte of interest. After an incubation period, this lane can be washed to prevent non-specific signals. Flow lanes below the second flow lane from the top are separately filled with antibody-conjugated catalase (antibody-catalase) and the catalase substrate, peroxide (H₂O₂). In the presence of catalase, H₂O₂ is broken into H₂O and O₂(g). Separate flow channels are formed when one plate is slid relative to the other. Formation of the flow channels causes the components of the flow lanes to mix. The antibody-catalase binds to the analyte of interest and the catalase reaction ensues. The O₂(g) released by the reaction causes the ink/dye in the top lanes to flow upwards through the flow channels. The more O₂(g) produced, which is dependent upon the amount of cytokine bound to the antibodies, the further the ink/dye will travel.

### Methods of Managing Clinical Progression

As noted above, the present technology provides methods for managing the clinical progression of osteoarthritis in a mammalian subject, comprising:
(a) measuring the concentration, in the blood of the subject, of a cytokine selected from the group consisting of platelet-derived growth factor AB (PDGF-AB), platelet-derived growth factor BB (PDGF-BB), epidermal growth factor (EGF), and combinations thereof;
(b) comparing the concentration of the cytokine to a cytokine reference level to determine a cytokine level variance; and
(c) initiating a clinical action based on the cytokine level variance.
   In accordance with the method, the clinical action can comprise administration of a treatment regimen, modifying the timing of a treatment regimen, modifying the level of a treatment regimen, terminating a treatment regimen, and combinations thereof. Treatment regimens include any methods known in the art for the treatment or prevention of osteoarthritis, such as administration of systemic or topical drugs, biologics, and physical therapy. Drug therapies include administration of non-steroidal anti-inflammatories, steroids, and combinations thereof.

In some embodiments, the treatment regimen comprises administration of an anti-inflammatory composition comprising interleukin-1 receptor antagonist (IL-1ra) and soluble tumor necrosis factor receptor I (sTNF-RI). The anti-inflammatory composition can be a Protein Solution comprising IL-1ra at a concentration of at least about 10,000 pg/ml, sTNF-RI at a concentration of at least about 1,200 pg/ml, and a protein selected from the group consisting of sTNF-RII, IGF-I, EGF, HGF, PDGF-AB, PDGF-BB, VEGF, TGF-β1, and sIL-1RII, and mixtures thereof, wherein the concentration of the protein in the composition is greater than the concentration of the protein in normal blood. The Protein Solution may be from either autologous or allogeneic sources, relative to the subject.

Protein Solutions may be made by derivation of one or more components from white blood cells, adipose stromal cells, bone marrow stromal cells, or other tissue comprising cytokine-producing cells. In some embodiments, methods comprise fractionating a liquid (a “cytokine cell suspension.”) comprising cells capable of producing cytokines, such as IL1-ra and sTNF-R1. Methods may comprise fractionating a cytokine cell suspension by contacting the liquid, such as platelet rich plasma, with a solid extraction material, such as polyacrylamide beads. Protein Solutions and methods of making Protein Solutions among those useful herein are described in U.S. Patent Application Publication No. 2009/0220482, Higgins et al., published Sep. 3, 2009; U.S. Patent Application Publication No. 2010/0055087, Higgins et al., published Mar. 4, 2010; U.S. Patent Application Publication 2011/0052561, Hoeppner, published Mar. 3, 2011; International Application Publication 2012/030593, Higgins et al., published Mar. 8, 2012; and U.S. Patent Application Publication 2012/0172836, Higgins et al., published Jul. 5, 2012. Compositions and methods useful in aspects of the present technology are also described in the following applications filed Mar. 15, 2013: U.S. patent application Ser. No. 13/840,562, Binder et al., Methods and Non-Immunogenic Compositions for Treating Inflammatory Diseases; U.S. patent application Ser. No. 13/841,083, Landrigan, et al., Treatment of Inflammatory Respiratory Disease Using Protein Solutions; U.S. patent application Ser. No. 13/837,005, Woodell-May et al., Methods and Acellular Compositions for Treating Inflammatory Disorders; U.S. patent application Ser. No. 13/837,480, O'Shaughnessey, et al., Treatment of Pain Using Protein Solutions; U.S. patent application Ser. No. 13,839,280, Leach et al., Methods for Making Cytokine Compositions from Tissue Using Non-Centrifugal Methods; and U.S. patent application Ser. No. 13/840,129, Matusuka, et al., Treatment of Collagen Defects Using Protein Solutions; and U.S. patent application Ser. No. 13/841,103, Landrigan et al., Treatment of Peripheral Vascular Disease Using Protein Solutions; all of which are incorporated by reference herein.

In various embodiments, the Protein Solution comprises at least two proteins selected from the group consisting of IL-1ra (interleukin-1 receptor antagonist), sTNF-RI, sTNF-RII (soluble tumor necrosis factor-receptor 2), IGF-I (insulin-like growth factor 1), EGF (epidermal growth factor), HGF (hepatocyte growth factor), PDGF-AB (platelet-derived growth factor AB), PDGF-BB (platelet-derived growth factor BB), VEGF (vascular endothelial growth factor), TGF-β1 (transforming growth factor-β1, and sIL-1RII (soluble interleukin 1 receptor II), wherein the concentration of each protein in the composition is greater than the concentration of the protein in normal blood. While the concentration of every such protein in the composition may be greater than its respective concentrations in in normal blood, it is not necessary that the concentration of more than two of the proteins be greater than their respective concentrations in normal blood.

In various embodiments, a Protein Solution comprises the following components.

TABLE 1
Protein Solution Exemplary Protein Components

| | | Normal Whole Blood |
|---|---|---|
| | Composition Concentration | Concentration |
| Component | | |
| plasma | about 80 mg/ml or greater | about 67 mg/ml |
| proteins | about 100 mg/ml or greater | |
| (total) | about 200 mg/ml or greater | |
| | about 250 mg/ml or greater | |
| albumin | about 60 mg/ml or greater | about 56 mg/ml |
| | about 100 mg/ml of greater | |
| fibrinogen | about 3.2 mg/ml or greater | about 2.9 mg/ml |
| | about 4 mg/ml or greater | |
| IL-1ra | about 10,000 pg/ml or greater | about 4200 pg/ml |
| | about 25,000 pg/ml or greater | |
| | about 30,000 pg/ml or greater | |
| | from about 25,000 to about | |
| | 110,000 pg/ml | |
| | from about 25,000 to about | |
| | 40,000 pg/ml | |
| sTNF-RI | about 1,200 pg/ml or greater | about 630 pg/ml |
| | about 1,800 pg/ml or greater | |
| | about 3,000 pg/ml or greater | |
| sTNF-RII | about 3,000 pg/ml or greater | about 1200 pg/ml |
| | about 5,000 pg/ml or greater | |
| | about 7,000 pg/ml or greater | |
| | about 9,000 pg/ml or greater | |
| sIL-1RII | about 15,000 pg/ml or greater | about 11,800 pg/ml |
| | about 20,000 pg/ml or greater | |
| | about 25,000 pg/ml or greater | |
| Growth | | |
| factors | | |
| EGF | about 800 pg/ml or greater | about 250 pg/ml |
| | about 1,000 pg/ml or greater | |
| | about 1,200 pg/ml or greater | |
| HGF | about 1,000 pg/ml or greater | about 500 pg/ml |
| | about 2,500 pg/ml or greater | |
| | about 2,800 pg/ml or greater | |
| | about 3,000 pg/ml or greater | |
| PDGF-AB | about 35,000 pg/ml or greater | about 6,000 pg/ml |
| | about 50,000 pg/ml or greater | |
| | about 70,000 pg/ml or greater | |
| PDGF-BB | about 10,000 pg/ml or greater | about 1,500 pg/ml |
| | about 15,000 pg/ml or greater | |
| | about 20,000 pg/ml or greater | |
| TGF-β1 | about 100,000 pg/ml or greater | about 10,000 pg/ml |
| | about 150,000 pg/ml or greater | |
| | about 190,000 pg/ml or greater | |
| IGF-1 | about 130,000 pg/ml or greater | about 70,000 pg/ml |
| | about 150,000 pg/ml or greater | |
| | about 160,000 pg/ml or greater | |
| VEGF | about 500 pg/ml or greater | about 150 pg/ml |
| | about 600 pg/ml or greater | |
| | about 800 pg/ml or greater | |

Protein concentrations can be measured using the methods set forth in Example 4.

The composition further preferably comprises viable cytokine-producing cells, lysed cytokine-producing cells, or both. In a preferred composition, the Protein Solution comprises monocytes, granulocytes, and platelets. In various embodiments, a Protein Solution comprises the following components.

TABLE 2
Protein Solution Exemplary Cellular Components

| | | Normal Whole Blood | |
|---|---|---|---|
| Component | Composition Concentration | Concentration | |
| cytokine- | at least about 15 k/μl | 6.5 | k/μl |
| producing | at least about 30 k/μl | | |
| cells | from about 30 to about 60 k/μl | | |
| | from about 40 to about 50 k/μl | | |
| red blood | less than about 3M/μl | 4.5M/μl | |
| cells | less than about 2M/μl | | |
| | less than about 2.5M/μl | | |
| platelets | at least about 400 k/μl | 240 | k/μl |
| | at least about 800 k/μl | | |
| | at least about 1,000 k/μl | | |
| neutrophils | at least about 5 k/μl | 3.7 | k/μl |
| | at least about 10 k/μl | | |
| | at least about 12 k/μl | | |
| monocytes | at least about 1 k/μl | 0.5 | k/μl |
| | at least about 2 k/μl | | |
| | at least about 3 k/μl | | |
| lymphocytes | at least about 5 k/μl | 2 | k/μl |
| | at least about 10 k/μl | | |
| | at least about 20 k/μl | | |
| eosinophiles | at least about 0.15 k/μl | 0.1 | k/μl |
| | at least about 0.18 k/μl | | |
| basophils | at least about 0.2 k/μl | 0.1 | k/μl |
| | at least about 0.4 k/μl | | |
| | at least about 0.6 k/μl | | |

It will be understood that this concentration is species specific. Further, it is understood that concentrations may vary among individual subjects. Thus, in methods comprising production of a Protein Solution from the blood or other tissue containing cytokine-producing cells, the concentration of proteins and cells in the Protein Solution may vary from those recited above; the values recited above are mean values for concentrations as may be seen in a population of subjects.

In various embodiments, the concentration of one or more of the proteins or other components in the Protein Solution is greater than the concentration of the component in normal blood. (Compositions with such higher concentrations of components are said to be “rich” in such components.) As referred to herein, the concentration of a component in “normal” blood or other tissue is the concentration found in the general population of mammalian subjects from which the tissue is obtained, e.g., in normal whole blood. In methods wherein the anti-inflammatory cytokine composition is derived from tissue from a specific subject, the “normal” concentration of a protein or cell may be the concentration in the blood of that individual before processing is performed to derive the protein or cell.

Thus, in various embodiments, the concentration of one or more components of the Protein Solution is greater than about 1.5 times, about 2 times, or about 3 times, greater than the concentration of the component in normal blood. For example, components may have greater concentrations in the compositions, relative to normal (whole) blood, as follows:
IL-1ra, at a concentration that is at least about 2.5, or at least about 3 or at least about 5, times greater;
sTNF-RI, at a concentration that is at least about 2, or at least about 2.5 or at least about 3, times greater;
sTNF-RII, at a concentration that is at least about 2, or at least about 2.5 or at least about 3, times greater;
sIL-1RII, at a concentration that is at least about 1.5, or at least about 1.8 or at least about 2, times greater;
EGF, at a concentration that is at least about 2, or at least about 3 or at least about 5, times greater;
HGF, at a concentration that is at least about 2, or at least about 3 or at least about 4, times greater;
PDGF-AB, at a concentration that is at least about 2, or at least about 3 or at least about 5, times greater;
PDGF-BB, at a concentration that is at least about 2, or at least about 3 or at least about 5, times greater;
TGF-β1, at a concentration that is at least about 3, or at least about 4 or at least about 6, times greater;
IGF-1, at a concentration that is at least about 1.2, or at least about 1.4 or at least about 1.5, times greater;
VEGF, at a concentration that is at least about 2, or at least about 2.5 or at least about 3, times greater;
cytokine-producing cells, at a concentration that is at least about 2, or at least about 3 or at least about 4, times greater;
platelets, at a concentration that is at least about 2, or at least about 3 or at least 4, times greater;
neutrophils, at a concentration that is at least 1.5, or at least 2 or at least 3, times greater;
monocytes, at a concentration that is at least 3, or at least 4 or at least 6, times greater;
lymphocytes, at a concentration that is at least 5, or at least 8 or at least 10, times greater; and
basophils, at a concentration that is at least 2, or at least 4 or at least 6, times greater
   Also, the concentration of erythrocytes in the Protein Solution is preferably at least half, or at least a third, of the centration of erythrocytes in normal blood.

For example, a Protein Solution may comprise:
(a) at least about 10,000 pg/ml IL1-ra;
(b) at least about 1,200 pg/ml sTNF-RI; and
(c) a protein selected from the group consisting of sTNF-RII, IGF-I, EGF, HGF, PDGF-AB, PDGF-BB, VEGF, TGF-β1, and sIL-1RII, and mixtures thereof, wherein the protein has a concentration higher than the protein's baseline concentration in normal blood. In another example, a Protein Solution comprises:
(a) interleukin-1 receptor antagonist (IL-1ra), at a concentration at least 3 times greater than the concentration of IL-1ra in normal blood;
(b) soluble tissue necrosis factor-r1 (sTNF-r1), at a concentration at least 2 times greater than the concentration of IL-1ra in normal blood;
(c) cytokine-producing cells at a concentration at least 2 times greater than the concentration of cytokine-producing cells in normal blood; and
(d) platelets, at a concentration at least 2 times greater than the concentration of platelets in normal blood.

In some embodiments, the concentration of IL-1ra in the Protein Solution is preferably at least 5,000, or at least 10,000, times greater than the concentration of interleukin-1α in the Protein Solution. The ratio of IL-1ra:interleukin-1β (IL-1β) concentrations is preferably at least 100. In some embodiments, the concentration of IL-1ra in the Protein Solution is preferably at least 1500, or at least 8000, times greater than the concentration of IL-1β in the Protein Solution. The ratio of sIL-1RII:interleukin-1β (IL-1β) concentrations is preferably greater than 1. In some embodiments, the sIL-1RII in the Protein Solution is preferably at least 2000, or at least 45000, times greater the concentration of interleukin-1β in the Protein Solution.

### Example

The methods of the present technology are exemplified by the following study. In particular, whole blood and autologous Protein Solution from osteoarthritis patients are analyzed to determine interactions between cytokines and growth factors, co-morbidities, concomitant medications and osteoarthritis indicators.

An autologous Protein Solution (APS) containing high levels of anti-inflammatory cytokines and growth factors has been prepared and used to reduce the effects of corresponding inflammatory cytokines in vitro. Such methods and devices are described in U.S. Patent Application Publication No. 2009/0220482, and U.S. Patent Application Publication No. 2010/0055087, both of which are incorporated by reference herein. Although all research to date has been performed using blood from a relatively healthy donor population, it has been hypothesized that anti-inflammatory cytokines can be captured equally well from the blood of osteoarthritis patients. The purpose of this study was to determine interactions of cytokines and growth factors, medications, and co-morbidities in osteoarthritis blood and the effect on APS composition.

Blood from 105 patients with evidence of radiographic knee osteoarthritis was collected and processed to create APS. Patient medical history was collected as well as responses to Knee injury and Osteoarthritis Outcome Score (KOOS) surveys. Analysis of cytokines and growth factors in baseline and APS samples were performed using enzyme-linked immunosorbent assay (ELISA). Spearman Rank correlation was used to identify relationships between cytokines and growth factors, co-morbidities, medications and osteoarthritis indicators. IL-1 receptor antagonist (IL-1ra), soluble IL-1 receptor II (sIL-1RII) and soluble TNF receptors I and II (sTNF-RII, sTNF-RII) were captured in APS from osteoarthritis patients at least as well as from healthy donors. See Table 3, below.

TABLE 3
Baseline and APS concentrations of key cytokines
from osteoarthritis patients and healthy donors

| | | Baseline Conc. | APS Conc. | Fold |
|---|---|---|---|---|
| Cytokine | | (pg/ml) | (pg/ml) | Increase |
| IL-1β | Healthy | 3.4 | 3.8 | 1.1 |
| | OA | 3.3 | 8.9 | 2.7 |
| IL-1ra | Healthy | 8092 | 30853 | 3.8 |
| | OA | 7576 | 41896 | 5.5 |
| sIL-1RII | Healthy | 7361 | 20477 | 2.8 |
| | OA | 9535 | 20573 | 2.2 |
| TNF-α | Healthy | ND | 3.4 | ND |
| | OA | ND | 4.3 | ND |
| sTNF-RI | Healthy | 629 | 2408 | 3.8 |
| | OA | 808 | 3011 | 3.7 |
| sTNF-RII | Healthy | 2485 | 9491 | 3.8 |
| | OA | 1491 | 5060 | 3.4 |

Patients who took glucosamine/chondroitin supplements and propionic acid derivatives (e.g., ibuprofen) exhibited lower circulating levels of sTNF-R and IL-1ra. See Table 4, below.

TABLE 4
Positive (+) and Negative (−) Factors affecting concentrations
of key cytokines in circulating blood of osteoarthritis patients

| | | | | ILra:IL1β |
|---|---|---|---|---|
| TNFα | sTNF-R | IL-1β | IL-1ra | Ratio |
| (−) | (+) | (−) | (+) | (−) |
| diuretic | angiotensin II | high | angiotensin II | BMI |
| medica- | receptor | cholesterol/ | receptor | |
| tion | antagonist | statins | antagonist | |
| | (−) | (−) | (+) | (−) |
| | glucosamine | multi- | smoking | subchondral |
| | chondroitin | vitamins | | sclerosis |
| | (−) | | (−) | |
| | propionic | | age | |
| | acid | | | |
| | derivative | | | |
| | | | (−) | |
| | | | glucosamine | |
| | | | chondroitin | |
| | | | (−) | |
| | | | propionic | |
| | | | acid | |
| | | | derivative | |

Circulating platelet-derived growth factor AB (PDGF-AB) concentration was found to correlate with all measured osteoarthritis indicators: joint space narrowing, subchondral sclerosis, subchondral cysts and osteophytes, indicating it may be a potential predictor of disease severity. See Table 5, below.

TABLE 5
Factors exhibiting negative (−) and positive (+) relationship
with indicators of osteoarthritis

| Joint Space | | Subchondral | Subchondral |
|---|---|---|---|
| Narrowing | Osteophytes | Sclerosis | Cysts |
| (−) | (−) | (−) | (−) |
| PDGF-AB | PDGF-AB | PDGF-AB | PDGF-AB |
| (+) | (+) | (−) | (−) |
| age | vitamin D | IFG-1 | EGF |
| (++) | | | |
| thyroid | | | |
| medication | | | |
| | | (−) | (−) |
| | | IL-1ra:IL1β ratio | thyroid |
| | | | medication |
| | | (+) | |
| | | high cholesterol, | |
| | | thyroid medication, | |
| | | NSAIDs, nutritional | |
| | | supplements | |

Factors such as age, gender, and multivitamins can affect patients' KOOS responses. See Table 6, below.

TABLE 6
Positive (+) and negative (−) effects on KOOS responses.

| | | | | Muscle | Nutritional | Vitamin D |
|---|---|---|---|---|---|---|
| | | Gender | Sensi- | Relax- | Supple- | Supple- |
| | Age | (Female) | tizers | ants | ments | ments |
| KOOS | + | | + | − | | + |
| Symptom | | | | | | |
| KOOS | + | | + | − | | |
| Pain | | | | | | |
| KOOS | | − | | − | + | |
| Daily Life | | | | | | |
| KOOS | + | − | | | | |
| Sports | | | | | | |
| KOOS | + | | | − | + | |
| Quality of | | | | | | |
| Life | | | | | | |
| KOOS | + | − | | − | + | |
| Overall | | | | | | |

Circulating TNF-α correlated most with IL-1β concentration. Patients who took glucosamine/chondroitin supplements showed lower circulating levels of the antagonists soluble tumor necrosis factor receptor (sTNF-R) and IL-1 receptor antagonist (IL-1ra). Circulating platelet-derived growth factor AB (PDGF-AB) concentration was found to correlate with the osteoarthritis indicators subchondral sclerosis, subchondral cysts, and osteophytes indicating it may be a potential predictor of disease severity. Circulating concentration of PDGF-AB may be able to provide a diagnostic measure of osteoarthritis severity.

### NON-LIMITING DISCUSSION OF TERMINOLOGY

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The disclosure of all patents and patent applications cited in this disclosure are incorporated by reference herein.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

1. A method for assessing osteoarthritis in a mammalian subject, comprising
(a) measuring, in a tissue sample of the subject, the concentration of a cytokine selected from the group consisting of platelet-derived growth factor AB (PDGF-AB), platelet-derived growth factor BB (PDGF-BB), epidermal growth factor (EGF), and combinations thereof;
(b) comparing the concentration of the cytokine in the tissue sample of the subject to a cytokine reference level; and
(c) accessing osteoarthritis based on the result of the comparison; wherein
(d) the cytokine reference level of PDGF-AB at a whole blood concentration is from about 2 to about 32 ng/ml, the cytokine reference level of PDGF-BB at a whole blood concentration is from about 1 to about 16 ng/ml, and the cytokine reference level of EGF at a whole blood concentration is from about 87 to about 1300 pg/ml.

2-6. (canceled)

7. The method of claim 1, wherein the tissue sample is whole blood, platelet rich plasma, or platelet poor plasma.

8. (canceled)

9. The method of claim 1, wherein the cytokine reference level is a mean or median value of tissue sample concentrations of the cytokine in a population of the mammalian subjects not having osteoarthritis.

10. (canceled)

11. The method of claim 1, wherein the cytokine is PDGF-AB, the tissue sample is whole blood, platelet rich plasma, or platelet poor plasma, and the cytokine reference level of PDGF-AB at a whole blood concentration is from about 6 to about 17 ng/ml.

12. The method of claim 1, wherein the cytokine is PDGF-BB, the tissue sample is whole blood, platelet rich plasma, or platelet poor plasma, and the cytokine reference level of PDGF-BB at a whole blood concentration is about 1.9 to about 7 ng/ml.

13. The method of claim 1, wherein the cytokine is EGF, the tissue sample is whole blood, platelet rich plasma, or platelet poor plasma, and the cytokine reference level of EGF at a whole blood concentration is about 110 to about 420 pg/ml.

14. (canceled)

15. (canceled)

16. The method of claim 1, for monitoring the progression of osteoarthritis in the subject, wherein the cytokine reference level is the concentration of the cytokine in tissue sample obtained from the subject at a time prior to the time of the measuring, wherein the time prior is at least about 4 weeks, or at least about 3 months, or at least about 6 months, or at least about one year.

17-26. (canceled)

27. The method of claim 1, wherein the subject is a human.

28. The method of claim 1, wherein the subject is a companion, working, or sports animal.

29. A method of assessing osteoarthritis in a mammalian subject, comprising:
(a) measuring the level of a cytokine in a first sample of blood obtained from the subject;
(b) measuring the level of the cytokine in a second sample of blood obtained from the subject; and
(c) comparing the level of the cytokine in the first sample with the level of the cytokine in the second sample to determine a cytokine level change; wherein
(d) the cytokine is selected from the group consisting of platelet-derived growth factor AB (PDGF-AB), platelet-derived growth factor BB (PDGF-BB), epidermal growth factor (EGF), and combinations thereof.

30. The method of claim 29, for monitoring the progression of osteoarthritis in a mammalian subject, further comprising:
(i) conducting a first radiographic assessment of a joint of the subject at the time of measuring the level of the cytokine in the first sample;
(ii) conducting a second radiographic assessment of the joint of the subject at the time of measuring the level of the cytokine in the second sample;
(iii) comparing the first radiographic assessment to the second radiographic assessment to determine a radiographic change; and
(iv) correlating the radiographic change to the cytokine level change.

31. (canceled)

32. A method of managing the clinical progression of osteoarthritis in a mammalian subject, comprising:
(a) measuring the concentration, in the blood of the subject, of a cytokine selected from the group consisting of platelet-derived growth factor AB (PDGF-AB), platelet-derived growth factor BB (PDGF-BB), epidermal growth factor (EGF), and combinations thereof;
(b) comparing the concentration of the cytokine to a cytokine reference level to determine a cytokine level variance; and
(c) initiating a clinical action based on the cytokine level variance.

33. The method of claim 32, wherein the clinical action is selected from the group of initiating administration of a treatment regimen, modifying the timing of a treatment regimen, modifying the level of a treatment regimen, terminating a treatment regimen, and combinations thereof.

34. The method of claim 33, wherein the treatment regimen comprises administration of an anti-inflammatory composition comprising interleukin-1 receptor antagonist (IL-1ra) and soluble tumor necrosis factor receptor I.

35. The method of claim 34, wherein the anti-inflammatory composition comprises:
interleukin-1 receptor antagonist (IL-1 ra) at a concentration of at least about 10,000 pg/ml;
(ii) soluble Tumor Necrosis Factor Receptor 1 (sTNF-R1) at a concentration of at least about 1,200 pg/ml; and
(iii) a protein selected from the group consisting of sTNF-RII, IGF-I, EGF, HGF, PDGF-AB, PDGF-BB, VEGF, TGF-1β, and sIL-1RII, and mixtures thereof, wherein the concentration of the protein in the composition is greater than the concentration of the protein in normal blood.

36. (canceled)

37. (canceled)

38. The method of claim 32, wherein the cytokine reference level is the concentration of the cytokine in blood obtained from the subject at a time at least 4 weeks prior to the time of the measuring.

39. (canceled)

40. (canceled)

41. The method of claim 32, wherein the cytokine reference level is a mean or median value of blood concentrations of the cytokine in a population of the mammalian subjects not having osteoarthritis.

42-46. (canceled)

47. The method of claim 32, wherein the subject has an osteoarthritis symptom selected from the group consisting of joint pain, joint stiffness, joint injury, radiographic anomaly, and combinations thereof.

48-50. (canceled)

51. The method of claim 32, wherein the cytokine reference level of PDGF-AB at a whole blood concentration is from about 6 to about 17 ng/ml.

52. The method of claim 32, wherein the cytokine reference level of PDGF-BB at a whole blood concentration is about 2 to about 7 ng/ml.

53. The method of claim 32, wherein the cytokine reference level of EGF at a whole blood concentration is about 110 to about 420 pg/ml.
